(19) United States
(12) Patent Application Publication
(10) Pub. No.: US 2022/0037026 A1
(43) Pub. Date: Feb. 3, 2022

(54) SYSTEM AND METHOD FOR PERSONALIZED TRIAGE WITH SURVIVAL MODELING AND CONSTRAINED OPTIMIZATION

(71) Applicant: KONINKLIJKE PHILIPS N.V., Eindhoven (NL)

(72) Inventors: Yale Chang, Lincoln, MA (US); Shruti Gopal Vij, Cambridge, MA (US); Lasith Adhikari, Revere, MA (US)

(21) Appl. No.: 17/236,345

(22) Filed: Apr. 21, 2021

Related U.S. Application Data
(60) Provisional application No. 63/057,330, filed on Jul. 28, 2020.

(51) Int. Cl. G16H 50/30; G06N 20/00; G06N 5/04; G06N 7/00; G16H 50/70; G16H 20/00; G16H 40/20; G16H 50/20

(52) U.S. Cl. CPC G16H 50/30; G06N 20/00; G06N 5/04; G16H 50/20; G16H 50/70; G16H 20/00; G16H 40/20; G06N 7/005

(57) ABSTRACT

A method for performing, using a victim triage system, triage analysis of victims of an incident, comprising: (i) receiving a location of the incident, medical information, hospital capability information for hospitals in a predetermined vicinity of the location, and transport information relative to the location; (ii) determining, by a trained triage machine learning algorithm using the received information, a triage decision for the victims, wherein the triage decision for a victim comprises: (1) a probability of the victim's survival over time; (2) a recommendation to transport or not transport the victim to a hospital; and (3) to which of the two or more hospitals the victim should be transported; (iii) generating (140) a triage report comprising the determined triage decision for each of the plurality of victims; and (iv) displaying the triage report on a user display of the victim triage system.

### FIELD OF THE DISCLOSURE

The present disclosure is directed generally to methods and systems for maximizing survival of a plurality of victims of a mass casualty incident.

### BACKGROUND

Mass casualty incidents (MCI) are unpredictable and create an influx of patients to hospitals. In a study of mass casualty incidents in 2010 in the United States, 14,504 emergency medical services (EMS) responses were recorded from 9,913 unique MCIs. The study showed that victims of MCIs have a statistically higher chance of experiencing response delays and other system delays in being transported to hospitals and care facilities. As per the 2018 National Crime Victims' Rights Week Resource Guide: Crime and Victimization Fact Sheets, mass shootings are the most common mass casualty events in the United States. Between 1990 and 2017 there have been 87 recorded mass shootings in the United States more than half of these occurring between 2008 and 2017. Given the increase in the number of MCIs in the United States, a corresponding increase in interest in management techniques such as hospital drills and triage training has been observed including studies of institutional preparedness for MCIs.

Unfortunately, MCIs often overwhelm available material and human resources requiring an organized management of the patients even at the scene. Accordingly, MCI triage protocols are notably different from routine triage. Mass casualty triage systems are designed to prioritize victims in more severe condition to save as many victims as possible. For example, the Simple Triage and Rapid Treatment (START) system assigns victims to four severity levels based on victims' ability to walk, respirations, perfusion, and mental status. The triage protocol categorizes patients based on acuity and need for medical attention. The first victims to be transported are patients with critical injuries (red) and ones that are expected to deteriorate in a couple of hours (yellow). Low priority is given to patients with minor injuries (green) or dead victims (black). Other commonly used triage systems include JumpSTART (JumpSTART), SALT (SALT), CBRN and Emergency Severity Index. These triage systems however have many limitations including being a subjective assessment, having inadequate validation on real-world victim cohorts, and insufficient distribution of patients across severity levels. Validation studies showed their triage outcomes only had weak to modest correlation with the actual injury severity outcomes and almost half of emergency department (ED) patients nationally are categorized as yellow.

Machine learning-based triage algorithms have been developed to predict whether a patient would expire or be admitted to the intensive care unit at the end of the stay. These algorithms can automatically learn non-linear combinations of the patient's input variables to predict the clinical outcome and achieve superior performance compared to the ESI. The current state of the art machine learning algorithms in this space is limited to evaluate victims' current severity levels. However, different victims can have different rates of deterioration and many patients' severity status changes significantly in the duration they wait to be transported to care facilities. Furthermore, the triage decision is constrained by different capacities and transport times of multiple hospitals.

### SUMMARY OF THE DISCLOSURE

There is a continued need for triage methods and systems that provide a triage decision for a plurality of victims or patients based on the victims' or patients' predicted future severity status.

The present disclosure is directed at inventive methods and systems for generating a triage report comprising triage decisions for a plurality of victims. Various embodiments and implementations herein are directed to a system or method that comprises survival modeling to predict the victim's survival probability if being transported to the hospital, and generalized assignment optimization to maximize the expected number of survival victims under constraints. The system receives information relevant to a triage decision, including the location of the incident, medical information about the patients, hospital capability information for hospitals in the vicinity of the location of the incident, and transport information relative to the location of the incident. A trained triage machine learning algorithm uses the received information to generate a triage decision for each victim, comprising a probability of the victim's survival over time, a recommendation to transport or not transport the victim to a hospital; and a hospital to send the victim to. The system generates a triage report comprising the determined triage decision for each of the victims, and displays the report on a user display, including a display of the probability of the victim's survival over time and the victim's transport recommendation.

Generally, in one aspect, a method for triage analysis of a plurality of victims of an incident is provided. The method includes: (i) receiving a location of the incident; (ii) receiving medical information about the plurality of victims of the incident, the medical information comprising at least injury information for each of the plurality of victims; (iii) receiving hospital capability information for each of two or more hospitals in a predetermined vicinity of the location of the incident, wherein the hospital capability information comprises at least hospital capacity information and a capability of the hospital to treat the injury of one or more of the plurality of victims; (iv) receiving transport information relative to the location of the incident, wherein the transport information comprises at least a time until a transport arrives at the location of the incident; (v) determining, by a processor of the victim triage system comprising a trained triage machine learning algorithm and using the received location, medical information, hospital capability information, and transport information, a triage decision for each of the plurality of victims of the incident, wherein the triage decision for a victim comprises: a probability of the victim's survival over time; a recommendation to transport or not transport the victim to a hospital; and if transport to a hospital is recommended, to which of the two or more hospitals the victim should be transported to maximize survival of each of the plurality of victims for which transport is recommended; (vi) generating a triage report comprising the determined triage decision for each of the plurality of victims; and (vii) displaying the triage report on a user display of the victim triage system, comprising displaying for each victim the probability of the victim's survival over time and the victim's transport recommendation.

According to an embodiment, the method further includes training the triage machine learning algorithm, comprising: (1) receiving a dataset of incident victim data, the dataset comprising for each of a plurality of victims of an incident which were transported to a hospital: (i) the victim's feature vector x, (ii) an indictor of a fatal event for the victim, and (iii) the corresponding fatal event time T; (2) generating, from the dataset, a hazard function for each of the plurality of victims, wherein the hazard function comprises a risk of a fatal event for the victim at a time T; and (3) training the triage machine learning algorithm using the generated hazard functions for the plurality of victims.

According to an embodiment, the triage decision to which of the two or more hospitals the victim should be transported comprises an estimate of a time to arrival at each of the two or more hospitals.

According to an embodiment, the capability of the hospital to treat the injury of one or more of the plurality of victims comprises information about supply availability at the hospital and/or staff availability at the hospital.

According to an embodiment, a probability of the victim's survival (5) of being transported to a hospital (m) is determined using the equation S(xₙ, w+Tₘ), where xₙ is the victim's (n) features extracted from the received medical information about the victim, w is an estimated wait time until a transport arrival based on the received transport information, and Tₘ is a transport time to the hospital.

According to an embodiment, the method further includes receiving one or more of: (i) updated medical information about one or more of the plurality of patients, (ii) updated hospital capability information; and (iii) updated transport information; updating, by the trained triage machine learning algorithm, the triage decision for one or more of the plurality of victims; generating an updated triage report, comprising the updated triage decision for the one or more of the plurality of victims; and displaying the updated triage report on the user display.

According to an embodiment, the displayed updated triage report comprises an indication of a change between the original triage report and the updated triage report.

According to an embodiment, the method further includes the step of collecting vital data from each of the plurality of victims, wherein the medical information about each of the plurality of victims of the incident comprises the respective collected vital data.

According to an embodiment, the trained triage machine learning algorithm of the victim triage system is a cloud-based service.

According to another aspect, a victim triage system is provided. The system includes: (i) a classifier trained to generate a triage decision for each of a plurality of victims of an incident; (ii) medical information about the plurality of victims of the incident, the medical information comprising at least injury information for each of the plurality of victims; (iii) hospital capability information for each of two or more hospitals in a predetermined vicinity of a location of the incident, wherein the hospital capability information comprises at least hospital capacity information and a capability of the hospital to treat the injury of one or more of the plurality of victims; (iv) transport information relative to the location of the incident, wherein the transport information comprises at least a time until a transport arrives at the location of the incident; (v) a processor configured to: determine, using the classifier and using the medical information, hospital capability information, and transport information, a triage decision for each of the plurality of victims of the incident, wherein the triage decision for a victim comprises: a probability of the victim's survival over time; a recommendation to transport or not transport the victim to a hospital; and if transport to a hospital is recommended, to which of the two or more hospitals the victim should be transported to maximize survival of each of the plurality of victims for which transport is recommended; and generate a triage report comprising the determined triage decision for each of the plurality of victims; and (vi) a display configured to display the generated triage report, comprising a display for each victim the probability of the victim's survival over time and the victim's transport recommendation.

According to another aspect, a victim triage device is provided. The device includes: (i) a user interface configured to receive: a location of the incident; medical information about the plurality of victims of the incident, the medical information comprising at least injury information for each of the plurality of victims; hospital capability information for each of two or more hospitals in a predetermined vicinity of the location of the incident, wherein the hospital capability information comprises at least hospital capacity information and a capability of the hospital to treat the injury of one or more of the plurality of victims; and transport information relative to the location of the incident, wherein the transport information comprises at least a time until a transport arrives at the location of the incident; (ii) a classifier trained to generate a triage decision for each of the plurality of victims of the incident; (iii) a processor configured to: determine, using the classifier and using the location information, the medical information, the hospital capability information, and the transport information, a triage decision for each of the plurality of victims of the incident, wherein the triage decision for a victim comprises: a probability of the victim's survival over time; a recommendation to transport or not transport the victim to a hospital; and if transport to a hospital is recommended, to which of the two or more hospitals the victim should be transported to maximize survival of each of the plurality of victims for which transport is recommended; and generate a triage report comprising the determined triage decision for each of the plurality of victims; and (iv) a display configured to display the generated triage report, comprising a display for each victim the probability of the victim's survival over time and the victim's transport recommendation.

In various implementations, a processor or controller may be associated with one or more storage media (generically referred to herein as “memory,” e.g., volatile and non-volatile computer memory such as RAM, PROM, EPROM, and EEPROM, floppy disks, compact disks, optical disks, magnetic tape, etc.). In some implementations, the storage media may be encoded with one or more programs that, when executed on one or more processors and/or controllers, perform at least some of the functions discussed herein. Various storage media may be fixed within a processor or controller or may be transportable, such that the one or more programs stored thereon can be loaded into a processor or controller so as to implement various aspects as discussed herein. The terms “program” or “computer program” are used herein in a generic sense to refer to any type of computer code (e.g., software or microcode) that can be employed to program one or more processors or controllers.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

These and other aspects of the various embodiments will be apparent from and elucidated with reference to the embodiment(s) described hereinafter.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the various embodiments.

FIG. 1 is a flowchart of a method for perform triage analysis of a plurality of victims of an incident using a victim triage system, in accordance with an embodiment.

FIG. 2 is a schematic representation of a victim triage system, in accordance with an embodiment.

### DETAILED DESCRIPTION OF EMBODIMENTS

The present disclosure describes various embodiments of a system and method for generating a triage report comprising triage decisions for a plurality of victims. Applicant has recognized and appreciated that it would be beneficial to provide a method and system that can provide a triage decision for a plurality of victims or patients based on predicted future severity status. The system receives information relevant to a triage decision, including the location of the incident, medical information about the patients, hospital capability information for hospitals in the vicinity of the location of the incident, and transport information relative to the location of the incident. A trained triage machine learning algorithm uses the received information to generate a triage decision for each victim, comprising a probability of the victim's survival over time, a recommendation to transport or not transport the victim to a hospital, and a hospital to send the victim to. The system generates a triage report comprising the determined triage decision for each of the victims, and displays the report on a user display, including a display of the probability of the victim's survival over time and the victim's transport recommendation.

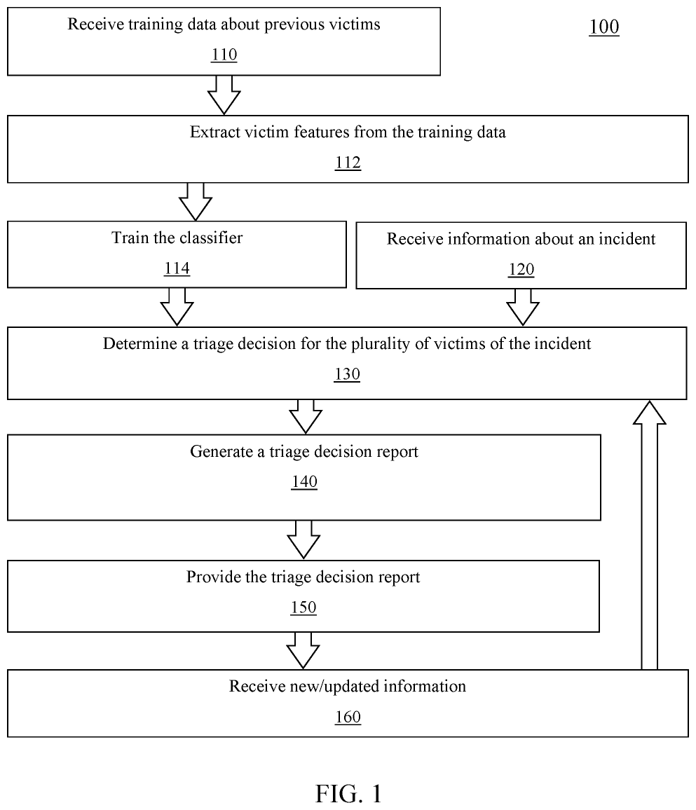

Referring to FIG. 1, in one embodiment, is a flowchart of a method 100 for performing, using a victim triage system, triage analysis of a plurality of victims of an incident to maximize survival of each of the victims. The victim triage system may be any victim triage system described or otherwise envisioned herein.

As discussed in greater detail herein, the victim triage system comprises a machine learning algorithm, also called a classifier, that has been trained to determine a probability of the victim's survival over time, which can be utilized to generate a recommendation to transport or not transport the victim to a hospital, and a hospital to send the victim to. The machine learning algorithm of the victim triage system can be trained using a dataset of historical victim data, the dataset comprising for each of a plurality of victims medical information about the victims, an indication of a fatal event or survival for the victim, and the time of the fatal event. Features are extracted from the historical victim medical information and utilized to train the machine learning algorithm of the victim triage system.

According to an embodiment, the victim triage system may be embodied in whole or in part within a device. For example, the entire victim triage system may be embodied within a single device such as a handheld device, laptop, computer, or other single device. Alternatively, the victim triage system may comprise a user interface that is transportable, such as a handheld device, mobile phone application, computer, or other transportable element that functions as a user interface to receive information at the incident. The device will communicate the information to the another, remote component of the victim triage system for analysis. The result of the victim triage system may then be communicated back to the transportable user interface.

At step 110 of the method, the victim triage system receives input data comprising training data about a plurality of victims in previous incidents. The training data can comprise medical information about each of the victims, including but not limited to demographics, physiological measurements such as vital data, injury information, physical observations, and/or diagnosis, among many other types of medical information. The medical information may include information obtained at the site of the incidence as well as medical information obtained during transport of the victim to a hospital, and/or medical information obtained about the victim at the hospital. As an example, the medical information can include detailed information on patient demographics such as age, gender, and more; diagnosis or medication condition such as cardiac disease, psychological disorders, chronic obstructive pulmonary disease, and more; physiologic vital signs such as heart rate, blood pressure, respiratory rate, oxygen saturation, and more; and/or physiologic data such as heart rate, respiratory rate, apnea, SpO₂, invasive arterial pressure, noninvasive blood pressure, and more. Many other types of medical information are possible.

The training data may also comprise an indication or information about whether the victim survived or died, how they died, where they died, and/or what time they died. For example, the training data may reveal that a victim suffering from some observed injuries was transported to a hospital 35 minutes away and died 20 minutes into transport. The training data may reveal that a victim suffering from some observed injuries was transported to a hospital 35 minutes away and survived.

This training data may be stored in and/or received from one or more databases. The database may be a local and/or remote database. For example, the victim triage system may comprise a database of training data.

According to an embodiment, the victim triage system may comprise a data pre-processor or similar component or algorithm configured to process the received training data. For example, the data pre-processor analyzes the training data to remove noise, bias, errors, and other potential issues. The data pre-processor may also analyze the input data to remove low quality data. Many other forms of data pre-processing or data point identification and/or extraction are possible.

At step 112 of the method, the system extracts victim features from the received training data. This can be accomplished by a variety of embodiments for feature identification, extraction, and/or processing, including any method for extracting features from a dataset. The outcome of a feature processing step or module of the victim triage system is a set of victim features related to medical information about a victim and survival time and/or outcome, which thus comprises a training data set that can be utilized to train the classifier.

At step 114 of the method, the system trains the machine learning algorithm, which will be the classifier utilized to analyze victim information as described or otherwise envisioned herein. The machine learning algorithm is trained using the extracted features according to known methods for training a machine learning algorithm.

According to an embodiment, time-to-event outcome such as time to death or hospital admission, is utilized to train a survival model S(x, t) to predict a victim's probability of survival over time, where x is the victim's features and t is time. To improve the accuracy of the prediction, the victim triage system applies machine learning and deep learning-based survival models, such as random survival forests, DeepSurv, and DeepHit to extract the non-linear relationship between the victim's variables x and the hazard function.

After training the survival model S(x, t), given the n-th victim xₙ, the estimated wait time until the transport arrival w, and the m-th hospital with transport time Tₘ, the n-th victim's survival probability if being transported to the m-th hospital can be predicted as S(xₙ, w+Tₘ). The prediction can also be dynamically updated before the transport arrival if new measurements were collected for the patient's variables xₙ. Compared to estimating victims' deteriorations using expert inputs in prior art triage methods, the survival models described or otherwise envisioned can utilize the time-to-event information to automatically estimate deteriorations in a data driven way and generate more accurate estimation.

According to an embodiment, the training data can comprise at least: (i) a patient's feature vector x; (ii) an indicator, such as a binary indicator, representing an event such as death has occurred for the patient; and (ii) the victim's corresponding event time T. Using this information, the survival function can be defined as the probability the event hasn't occurred at time t using the following equation:

S(t)=P(T>t) (Eq. 1)

The hazard function can also be defined as the risk of event at time t using the following equation:

$$λ ( t ) = {lim}_{δ \to 0} \frac{P ( t \leq T < t + δ \vert T \geq t )}{δ} = {lim}_{δ \to 0} \frac{S ( t + δ ) - S ( t )}{δ} = \frac{dS ( t )}{dt} \qquad ( Eq . 2 )$$

The proportional hazards model assumes the hazard function can be decomposed as the product of a baseline hazard function λ₀(t) and a risk function h(x) using the following equation:

λ(t|x)=λ₀(t)·e^{h(x)} (Eq. 3)

In DeepSurv, as just one non-limiting example of a deep learning-based survival model, the risk function is modeled as a deep neural network (DNN) parameterized by network weights θ using the following equation:

h(x)=DNN_{θ}(x) (Eq. 4)

According to an embodiment, the model can be trained by maximizing the partial likelihood with regard to the network weights θ. Many other modifications of this approach are possible.

Following step 114, the victim triage system comprises a trained classifier that can be utilized to classify victim status and provide a probability of the victim's survival over time. The trained classifier can be static such that it is trained once and is utilized for classifying. According to another embodiment, the trained classifier can be more dynamic such that it is updated or re-trained using subsequently available training data. The updating or re-training can be constant or can be periodic.

At step 120 of the method, an incident has occurred and there are a plurality of victims of the incident. The victim triage system will be utilized to generate triage decisions for the emergency responders, medical personnel, and/or other individuals at the scene of the incident. Accordingly, at step 120 of the method information is received by the victim triage system. This information can be provided to the victim triage system directly by the individuals at the scene of the incident. For example, the victim triage system may be or comprise a transportable user interface that is utilized by an individual at the scene of the incident. The information can be provided to the victim triage system indirectly by an individual communicating with someone at the scene of the incident. For example, an individual at the scene may communicate by phone with someone that is interfacing with the victim triage system.

According to an embodiment, the victim triage system receives location information about the incident. This may be provided by an individual at the scene of the incident, such as by entering an address into the user interface of the victim triage system. According to an embodiment, the victim triage system may obtain this information automatically. For example, the victim triage system may comprise a GPS or other method of obtaining location information. The victim triage system may be in communication with a database of incident reports and thus can extract or impute the location from these reports. Many other methods of obtaining a location of the incident are possible.

According to an embodiment, the victim triage system receives medical information about some or all of the plurality of victims of the incident. The medical information can be any type of medical information that may be relevant to incident triage.

receiving, by the victim triage system, medical information about the plurality of victims of the incident, the medical information comprising at least injury information for each of the plurality of victims;

The training data can comprise medical information about each of the victims, including but not limited to demographics, physiological measurements such as vital data, injury information, physical observations, and/or diagnosis, among many other types of medical information. The medical information may include information obtained at the site of the incidence as well as medical information obtained during transport of the victim to a hospital, and/or medical information obtained about the victim at the hospital. As an example, the medical information can include detailed information on patient demographics such as age, gender, and more; diagnosis or medication condition such as cardiac disease, psychological disorders, chronic obstructive pulmonary disease, and more; physiologic vital signs such as heart rate, blood pressure, respiratory rate, oxygen saturation, and more; and/or physiologic data such as heart rate, respiratory rate, apnea, SpO₂, invasive arterial pressure, noninvasive blood pressure, and more. Many other types of medical information are possible.

According to an embodiment, the victim triage system receives hospital capability information for each of two or more hospitals in a predetermined vicinity of the location of the incident. The hospital capability information, for example, can include information about hospital capacity and a capability of the hospital to treat the injury of one or more of the plurality of victims. Capacity information can be information about the ability of the hospital to accept new emergency patients from the incident. Capability information can be information about the ability of the hospital to handle or treat the injuries of one or more victims at the incident. For example, some locations may not be equipped to handle certain types of injuries or emergencies. Capacity and/or capability information may be pre-programmed into the victim triage system or it may be obtained periodically or continually by communication with a database or other source of this information. For example, hospitals may maintain a database of current capacity and capabilities, and the victim triage system may be configured to query that database to obtain that information.

The predetermined vicinity may be any range that is suitable for transporting a victim of an incident. This may be pre-programmed, such as a user or other entity defining an allowable distance, or may be based on information gleaned due to threshold parameters of the victim triage system. For example, the victim triage system may determine, based on the received location information, that there are 4 hospitals within 100 miles and that 2 of those hospitals are within 25 miles. The system may be programmed to only find hospitals within 100 miles, and to preferably select two or more hospitals as close to the incident as possible.

According to an embodiment, the victim triage system receives transport information relative to the location of the incident. The transport information comprises at least a time until a transport arrives at the location of the incident, and can further comprise an estimated time for the victim to be transported by the transport to the selected or recommended hospital. This information may be received by an individual at the incident in contact with transport such as ambulances heading to the incident, and be input into the system. Additionally or alternatively, the information may be received automatically by the system due to interface with a transport tracking system. For example, the transport vehicles which may include ambulances, helicopters, airplanes, and/or other transport vehicles may be equipped with GPS or other location devices that continually provide location information or provide location information in response to a query. The victim triage system can then receive or query for that location information.

The received information may be stored in one or more databases, which may be local and/or remote databases. For example, the victim triage system may comprise a database or other memory configured to store the data.

At step 130 of the method, the victim triage system utilizes the trained triage machine learning algorithm to analyze the received location, medical information, hospital capability information, and transport information. This results in the generation of a triage decision for one or more of the plurality of victims of the incident. The triage decision can comprise, for example, a probability of the victim's survival over time, and a recommendation to transport or not transport the victim to a hospital. If the recommendation is to transport the victim to a hospital, the triage decision can comprise an indication of which of the two or more hospitals the victim should be transported. Preferably, the victim triage system and the trained triage machine learning algorithm make triage decisions for the cohort of victims that maximizes the probability of survival for the most victims. According to an embodiment, the decision to which of the two or more hospitals the victim should be transported comprises an estimate of a time to arrival at each of the two or more hospitals.

According to an embodiment, in order to determine how to transport N victims {x₁, . . . , x_{N}} to M hospitals with capacities {C₁, . . . , C_{M}} and transport time {T₁, . . . , T_{M}}, the system is configured to maximize the expected number of survival victims at the time when all victims have been transported. Since each hospital has limited capacity, not all victims can be transported to a hospital. Therefore, the system can use m=0 to represent “no transport” and can be set T₀=max{T₁, . . . , T_{M}}. The optimization problem can be characterized as follows:

$$\begin{matrix}{max}_{Z : {z}_{n m} ∈ \{ 0 , 1 \}} {\sum }_{n = 1}^{N} {\sum }_{m = 0}^{M} S ( {x}_{n} , w + {T}_{m} ) {z}_{n m} & ( Eq . 5 ) \\ s . t . {\sum }_{m = 0}^{M} {z}_{n m} = 1 , ∀ n ∈ \{ 1 , … , N \} ; \\ {\sum }_{n = 1}^{N} {z}_{n m} \leq {C}_{m} , ∀ m ∈ \{ 1 , … , M \} & ( Eq . 6 )\end{matrix}$$

where zₘₘ=1 indicates the n-th victim is transported to the m-th hospital and zₙₘ=0 otherwise. The objective represents the expected number of survival victims when all victims have been either transported to hospitals or left without transport. The first constraint means each victim can only be transported to one hospital or left without transport. The second constraint means the total number of victims transported to a hospital cannot exceed its capacity. This is a standard generalized assignment problem is solved through an efficient approximation algorithm, among other approaches. After solving this optimization problem, the system can use zₙₘ to make the triage decision for the n-th victim: (1) don't transport if zₙ₀=1; or (2) transport to the m-th hospital if zₙₘ=1, mϵ{1, . . . , M}.

Thus, the system optimizes the triage decision of each individual victim and therefore can incorporate more flexible constraints, such as whether a patient can be transported to a hospital based on the availability of blood products. Furthermore, the system can explicitly incorporate constraints induced by the transport time and capacities of multiple hospitals.

At step 140 of the method, the system generates a triage report. The triage report comprises the determined triage decision for each victim. The triage report may comprise many different configurations and information. For example, the triage report may comprise the probability of the victim's survival over time, and the victim's transport recommendation. The transport recommendation may be a no transport recommendation, or a recommendation to transport the victim. The transport recommendation may further comprise an indication of which of two or more locations to transport the victim. The triage report may further comprise an estimated transport time for the victim to the recommendation location.

At step 150 of the method, the system displays the triage report on a display of the system. The display may comprise information about probability of the victim's survival over time, and the victim's transport recommendation. The display may further comprise an indication of which of two or more locations to transport the victim, and/or an estimated transport time for the victim to the recommendation location. Other information is possible. Alternatively, the triage report may be communicated by wired and/or wireless communication to another device. For example, the system may communicate the triage report to a mobile phone, computer, laptop, wearable device, and/or any other device configured to allow display and/or other communication of the triage report.

According to one possible embodiment, at step 160 of the method the system may received updated information. The updated information may be the received location, medical information, hospital capability information, and transport information. For example, the status of a victim may change for the better or the worse which indicates that a new triage decision may be necessary. That updated medical information, such as worsening vital signs or improved prognosis, may be provided to the system. As another example, the status of a hospital capability for one or the selected hospitals may change, due to an event such as capacity being filled by the incident, and that information may be provided to the system. As another example, a transport vehicle may become disabled or stuck in traffic, and that information may be provided to the system.

With the updated information, the system returns to step 130 of the method to update the triage decision for one or more of the victims using the trained triage machine learning algorithm. The system can then generate an updated triage report, comprising the updated triage decision for the one or more of the plurality of victims, and can display the updated triage report on the user display. According to an embodiment, the updated triage report may comprise information about what changed in the information and/or in the decision for a victim.

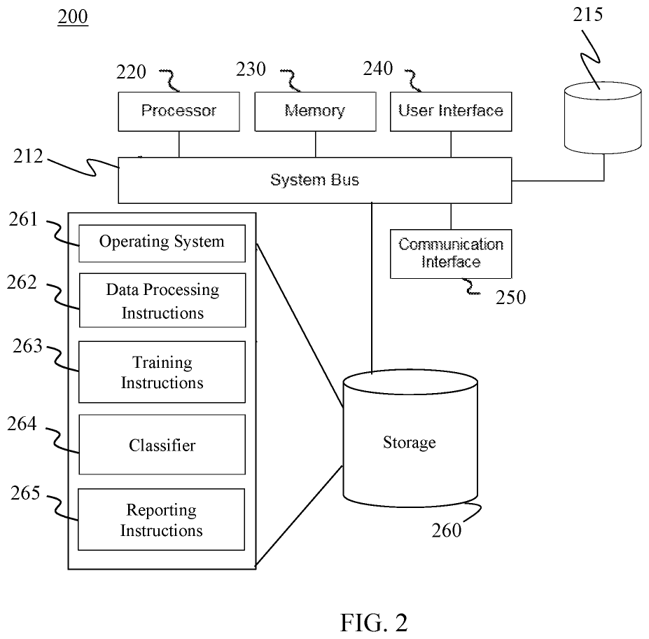

Referring to FIG. 2, in one embodiment, is a schematic representation of a victim triage system 200. System 200 may be any of the systems described or otherwise envisioned herein, and may comprise any of the components described or otherwise envisioned herein.

According to an embodiment, system 200 comprises one or more of a processor 220, memory 230, user interface 240, communications interface 250, and storage 260, interconnected via one or more system buses 212. It will be understood that FIG. 2 constitutes, in some respects, an abstraction and that the actual organization of the components of the system 200 may be different and more complex than illustrated.

According to an embodiment, system 200 comprises a processor 220 capable of executing instructions stored in memory 230 or storage 260 or otherwise processing data to, for example, perform one or more steps of the method. Processor 220 may be formed of one or multiple modules. Processor 220 may take any suitable form, including but not limited to a microprocessor, microcontroller, multiple microcontrollers, circuitry, field programmable gate array (FPGA), application-specific integrated circuit (ASIC), a single processor, or plural processors.

Memory 230 can take any suitable form, including a non-volatile memory and/or RAM. The memory 230 may include various memories such as, for example L1, L2, or L3 cache or system memory. As such, the memory 230 may include static random access memory (SRAM), dynamic RAM (DRAM), flash memory, read only memory (ROM), or other similar memory devices. The memory can store, among other things, an operating system. The RAM is used by the processor for the temporary storage of data. According to an embodiment, an operating system may contain code which, when executed by the processor, controls operation of one or more components of system 200. It will be apparent that, in embodiments where the processor implements one or more of the functions described herein in hardware, the software described as corresponding to such functionality in other embodiments may be omitted.

User interface 240 may include one or more devices for enabling communication with a user. The user interface can be any device or system that allows information to be conveyed and/or received, and may include a display, a mouse, and/or a keyboard for receiving user commands. In some embodiments, user interface 240 may include a command line interface or graphical user interface that may be presented to a remote terminal via communication interface 250. The user interface may be located with one or more other components of the system, or may located remote from the system and in communication via a wired and/or wireless communications network.

Communication interface 250 may include one or more devices for enabling communication with other hardware devices. For example, communication interface 250 may include a network interface card (NIC) configured to communicate according to the Ethernet protocol. Additionally, communication interface 250 may implement a TCP/IP stack for communication according to the TCP/IP protocols. Various alternative or additional hardware or configurations for communication interface 250 will be apparent.

Storage 260 may include one or more machine-readable storage media such as read-only memory (ROM), random-access memory (RAM), magnetic disk storage media, optical storage media, flash-memory devices, or similar storage media. In various embodiments, storage 260 may store instructions for execution by processor 220 or data upon which processor 220 may operate. For example, storage 260 may store an operating system 261 for controlling various operations of system 200.

It will be apparent that various information described as stored in storage 260 may be additionally or alternatively stored in memory 230. In this respect, memory 230 may also be considered to constitute a storage device and storage 260 may be considered a memory. Various other arrangements will be apparent. Further, memory 230 and storage 260 may both be considered to be non-transitory machine-readable media. As used herein, the term non-transitory will be understood to exclude transitory signals but to include all forms of storage, including both volatile and non-volatile memories.

While system 200 is shown as including one of each described component, the various components may be duplicated in various embodiments. For example, processor 220 may include multiple microprocessors that are configured to independently execute the methods described herein or are configured to perform steps or subroutines of the methods described herein such that the multiple processors cooperate to achieve the functionality described herein. Further, where one or more components of system 200 is implemented in a cloud computing system, the various hardware components may belong to separate physical systems. For example, processor 220 may include a first processor in a first server and a second processor in a second server. Many other variations and configurations are possible.

According to an embodiment, system 200 may comprise or be in remote or local communication with a database or data source 215. Database 215 may be a single database or data source or multiple. Database 215 may comprise the input data which may be used to train the classifier, as described and/or envisioned herein.

According to an embodiment, storage 260 of system 200 may store one or more algorithms and/or instructions to carry out one or more functions or steps of the methods described or otherwise envisioned herein. For example, processor 220 may comprise one or more of data processing instructions 262, training instructions 263, classifier 264, and/or reporting instructions 265.

According to an embodiment, data processing instructions 262 direct the system to retrieve and process input data which is used to either: (i) train the classifier 265 using the training instructions 263, or (ii) to perform a triage decision analysis for the victim using the trained classifier 264. The data processing instructions 262 direct the system to, for example, receive or retrieve input data or medical data to be used by the system as needed, such as from database 215 among many other possible sources. As described above, the input data can comprise a wide variety of input types from a wide variety of sources.

According to an embodiment, the data processing instructions 262 also direct the system to process the input data to generate a plurality of features related to medical information for a plurality of victims, which are used to train the classifier. This can be accomplished by a variety of embodiments for feature identification, extraction, and/or processing. The outcome of the feature processing is a set of features related to telemetry monitoring for a cohort of previously monitored patients, which thus comprises a training data set that can be utilized to train the classifier.

According to an embodiment, training instructions 263 direct the system to utilize the processed data to train the classifier to determine a triage decision for a victim, wherein the triage decision for a victim comprises a probability of the victim's survival over time. The classifier can be any machine learning classifier sufficient to utilize the type of input data provided. Thus, the system comprises a trained classifier 264 configured to determine a triage decision for a victim.

The trained classifier 264 is configured to determine a triage decision for a victim using the received location, medical information, hospital capability information, and transport information. The triage decision can comprise, for example, a probability of the victim's survival over time, and a recommendation to transport or not transport the victim to a hospital. If the recommendation is to transport the victim to a hospital, the triage decision can comprise an indication of which of the two or more hospitals the victim should be transported. Preferably, the victim triage system and the trained triage machine learning algorithm make triage decisions for the cohort of victims that maximizes the probability of survival for the most victims.

According to an embodiment, reporting instructions 265 direct the system to generate and provide a triage report. The triage report comprises the determined triage decision for each victim. The triage report may comprise many different configurations and information. For example, the triage report may comprise the probability of the victim's survival over time, and the victim's transport recommendation. The transport recommendation may be a no transport recommendation, or a recommendation to transport the victim. The transport recommendation may further comprise an indication of which of two or more locations to transport the victim. The triage report may further comprise an estimated transport time for the victim to the recommendation location. The reporting instructions 265 also direct the system to display the triage report on a display of the system. The display may comprise information about probability of the victim's survival over time, and the victim's transport recommendation. The display may further comprise an indication of which of two or more locations to transport the victim, and/or an estimated transport time for the victim to the recommendation location. Other information is possible. Alternatively, the triage report may be communicated by wired and/or wireless communication to another device. For example, the system may communicate the triage report to a mobile phone, computer, laptop, wearable device, and/or any other device configured to allow display and/or other communication of the triage report.

According to an embodiment, the communicated triage report can be utilized at the scene of the incident to enact one or more recommendations of the report, such as who to transport and where to transport them.

According to an embodiment, the victim triage system is configured to process many thousands or millions of datapoints in the input data used to train the classifier, as well as in the received location, medical information, hospital capability information, and transport information utilized for triage decisions for a plurality of victims of an incident. For example, generating a functional and skilled trained classifier using an automated process such as feature identification and extraction and subsequent training requires processing of millions of datapoints from input data and the generated features. This can require millions or billions of calculations to generate a novel trained classifier from those millions of datapoints and millions or billions of calculations. As a result, each trained classifier is novel and distinct based on the input data and parameters of the machine learning algorithm. Thus, generating a functional and skilled trained classifier comprises a process with a volume of calculation and analysis that a human brain cannot accomplish in a lifetime, or multiple lifetimes.

Similarly, the victim triage system can be configured to continually receive data about the cohort of victims, perform the analysis, and provide periodic or continual updates via the triage decision report for each victim. This requires the analysis of thousands or millions of datapoints on a continual basis to optimize the reporting, requiring a volume of calculation and analysis that a human brain cannot accomplish in a lifetime.

By providing a quick and thorough victim triage analysis, even in the midst of an incident that may could human judgment, this novel victim triage system has an enormous positive effect on triage compared to prior art systems. As just one example, by providing a system that can factor in the best possible outcome scenario for a plurality of victims of an incident, the system will improve the survival outcomes of the cohort and will lead to saved lives. The information needed by a human for adequate triage, particularly for a large cohort of victims, is likely to be incomplete due to the stress and activity of an incident, the numerous people involved in caring for victims, and other variables. Thus, triage by humans at an MCI is likely to be flawed compared to the triage performed by the novel systems and methods described or otherwise envisioned herein.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

What is claimed is:

1. A method for performing, using a victim triage system, triage analysis of a plurality of victims of an incident to maximize survival of each of the victims, comprising:
receiving, by the victim triage system, a location of the incident;
receiving, by the victim triage system, medical information about the plurality of victims of the incident, the medical information comprising at least injury information for each of the plurality of victims;
receiving, by the victim triage system, hospital capability information for each of two or more hospitals in a predetermined vicinity of the location of the incident, wherein the hospital capability information comprises at least hospital capacity information and a capability of the hospital to treat the injury of one or more of the plurality of victims;
receiving, by the victim triage system, transport information relative to the location of the incident, wherein the transport information comprises at least a time until a transport arrives at the location of the incident;
determining, by a processor of the victim triage system comprising a trained triage machine learning algorithm and using the received location, medical information, hospital capability information, and transport information, a triage decision for each of the plurality of victims of the incident, wherein the triage decision for a victim comprises: (1) a probability of the victim's survival over time; (2) a recommendation to transport or not transport the victim to a hospital; and (3) if transport to a hospital is recommended, to which of the two or more hospitals the victim should be transported to maximize survival of each of the plurality of victims for which transport is recommended;
generating, by the victim triage system, a triage report comprising the determined triage decision for each of the plurality of victims; and
displaying the triage report on a user display of the victim triage system, comprising displaying for each victim the probability of the victim's survival over time and the victim's transport recommendation.

2. The method of claim 1, further comprising the step of training the triage machine learning algorithm, comprising: (1) receiving a dataset of incident victim data, the dataset comprising for each of a plurality of victims of an incident which were transported to a hospital: (i) the victim's feature vector x, (ii) an indictor of a fatal event for the victim, and (iii) the corresponding fatal event time T; (2) generating, from the dataset, a hazard function for each of the plurality of victims, wherein the hazard function comprises a risk of a fatal event for the victim at a time T; and (3) training the triage machine learning algorithm using the generated hazard functions for the plurality of victims.

3. The method of claim 1, wherein the triage decision to which of the two or more hospitals the victim should be transported comprises an estimate of a time to arrival at each of the two or more hospitals.

4. The method of claim 1, wherein the capability of the hospital to treat the injury of one or more of the plurality of victims comprises information about supply availability at the hospital and/or staff availability at the hospital.

5. The method of claim 1, wherein a probability of the victim's survival (5) of being transported to a hospital (m) is determined using the equation S(xₙ, w+Tₘ), where xₙ is the victim's (n) features extracted from the received medical information about the victim, w is an estimated wait time until a transport arrival based on the received transport information, and Tₘ is a transport time to the hospital.

6. The method of claim 1, further comprising the step of:
receiving one or more of: (i) updated medical information about one or more of the plurality of patients, (ii) updated hospital capability information; and (iii) updated transport information;
updating, by the trained triage machine learning algorithm, the triage decision for one or more of the plurality of victims;
generating an updated triage report, comprising the updated triage decision for the one or more of the plurality of victims; and
displaying the updated triage report on the user display.

7. The method of claim 6, wherein the displayed updated triage report comprises an indication of a change between the original triage report and the updated triage report.

8. The method of claim 1, further comprising the step of collecting vital data from each of the plurality of victims, wherein the medical information about each of the plurality of victims of the incident comprises the respective collected vital data.

9. The method of claim 1, wherein the trained triage machine learning algorithm of the victim triage system is a cloud-based service.

10. A victim triage system configured to perform triage analysis of a plurality of victims of an incident to maximize survival of each of the victims, comprising:
a classifier trained to generate a triage decision for each of a plurality of victims of an incident;
medical information about the plurality of victims of the incident, the medical information comprising at least injury information for each of the plurality of victims;
hospital capability information for each of two or more hospitals in a predetermined vicinity of a location of the incident, wherein the hospital capability information comprises at least hospital capacity information and a capability of the hospital to treat the injury of one or more of the plurality of victims;
transport information relative to the location of the incident, wherein the transport information comprises at least a time until a transport arrives at the location of the incident;
a processor configured to: (i) determine, using the classifier and using the medical information, hospital capability information, and transport information, a triage decision for each of the plurality of victims of the incident, wherein the triage decision for a victim comprises: (1) a probability of the victim's survival over time; (2) a recommendation to transport or not transport the victim to a hospital; and (3) if transport to a hospital is recommended, to which of the two or more hospitals the victim should be transported to maximize survival of each of the plurality of victims for which transport is recommended; and (ii) generate a triage report comprising the determined triage decision for each of the plurality of victims; and
a display configured to display the generated triage report, comprising a display for each victim the probability of the victim's survival over time and the victim's transport recommendation.

11. The victim triage system of claim 10, wherein the triage decision to which of the two or more hospitals the victim should be transported comprises an estimate of a time to arrival at each of the two or more hospitals.

12. The victim triage system of claim 10, wherein the processor is further configured to: update, by the classifier, the triage decision for one or more of the plurality of victims based on one or more of updated medical information about one or more of the plurality of patients, updated hospital capability information; and updated transport information; and generate an updated triage report, comprising the updated triage decision for the one or more of the plurality of victims.

13. The victim triage system of claim 10, wherein the medical information comprises vital data collected from each of the plurality of victims.

14. The victim triage system of claim 10, wherein the classifier is cloud-based.

15. A victim triage device configured to perform triage analysis of a plurality of victims of an incident to maximize survival of each of the victims, comprising:
a user interface configured to receive: (i) a location of the incident; (ii) medical information about the plurality of victims of the incident, the medical information comprising at least injury information for each of the plurality of victims; (iii) hospital capability information for each of two or more hospitals in a predetermined vicinity of the location of the incident, wherein the hospital capability information comprises at least hospital capacity information and a capability of the hospital to treat the injury of one or more of the plurality of victims; and (iv) transport information relative to the location of the incident, wherein the transport information comprises at least a time until a transport arrives at the location of the incident;
a classifier trained to generate a triage decision for each of the plurality of victims of the incident;
a processor configured to: (i) determine, using the classifier and using the location information, the medical information, the hospital capability information, and the transport information, a triage decision for each of the plurality of victims of the incident, wherein the triage decision for a victim comprises: (1) a probability of the victim's survival over time; (2) a recommendation to transport or not transport the victim to a hospital; and (3) if transport to a hospital is recommended, to which of the two or more hospitals the victim should be transported to maximize survival of each of the plurality of victims for which transport is recommended; and (ii) generate a triage report comprising the determined triage decision for each of the plurality of victims; and
a display configured to display the generated triage report, comprising a display for each victim the probability of the victim's survival over time and the victim's transport recommendation.
